(19) United States
(12) Patent Application Publication
(10) Pub. No.: US 2009/0208939 A1
(43) Pub. Date: Aug. 20, 2009

(54) Identification of Molecular Diagnostic Markers for Endometriosis in Blood Lymphocytes

(76) Inventors: Idhaliz Flores, Coto Laurel, PR (US); Spyro Mousses, Scottsdale, AZ (US); Ester Rozenblum, Columbia, MD (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/884,402

(22) PCT Filed: Dec. 9, 2005
(86) PCT No.: PCT/US2005/044723

§ 371 Date: Jan. 7, 2009

Related U.S. Application Data
(60) Provisional application No. 60/654,331, filed on Feb. 18, 2005.

(51) Int. Cl. A61K 31/7052; C12Q 1/68; C12Q 1/02; G01N 33/53; A61P 15/00; G06F 19/00

(52) U.S. Cl. 435/6; 435/29; 435/7.24; 702/20

(57) ABSTRACT

The invention comprises a method of identifying or predicting the predisposition to endometriosis in a female subject comprising determining the level of gene expression of at least one differentially-expressed gene or protein or peptide of peripheral blood leukocytes in a sample of peripheral blood leukocytes or peripheral blood in a subject to provide a first value, determining the level of gene expression of the at least one differentially-expressed gene or protein or peptide of said leukocytes in a control or reference standard to provide a second value, and comparing whether there is a difference between the first value and second value.

### RELATED APPLICATIONS

This application claims benefit of U.S. Provisional Application No. 60/654,331 filed Feb. 18, 2005 which is hereby incorporated by reference in its entirety.

### FIELD OF THE INVENTION

Analysis of gene expression patterns of peripheral blood leukocytes of patients with endometriosis using DNA microarray-identified gene targets for non-invasive diagnostic assays for this disease.

### BACKGROUND OF THE INVENTION

Endometriosis is a fairly common gynecological disease, affecting as many as 10% of women in their reproductive years (Mahmood, T. A. and Templeton, A. 1991 Hum Reprod 6:544-549). The pathogenesis of endometriosis is still unknown, and the mechanisms whereby endometriotic lesions establish, progress, and migrate to extrapelvic sites are not well understood (Witz, C. A. et al. 2003 Hum Fertil 6:34-40). The diagnosis of endometriosis is generally carried out by visual inspection of the pelvis during laparoscopy (Attaran, M. et al. 2002 Cleve Clin J Med 69:647-653). This diagnostic procedure depends on the expertise of the surgeon and therefore it is intrinsically inaccurate. Also, the invasiveness and associated morbidity of the laparoscopic procedure precludes its use for monitoring recurrences and response to therapy. Despite the urgent need to diagnose endometriosis non-surgically and to have a non-invasive tool for prognosis and treatment monitoring, there are still no specific laboratory tests based on the identification of markers in blood (Falcone, T. and Mascha, E. 2003 Fertil Steril 80:886-888). Moreover, affected women who suffer chronic, severe pain, infertility, and mental anguish have few therapeutic options, none of which can cure the underlying disease. The search for candidate genes in which to base an endometriosis-specific test has proven to be challenging. Therefore, we aimed to use the DNA microarray technology to expedite the identification of molecular biomarkers for endometriosis.

DNA microarrays are increasingly being used to identify gene expression profiles associated with complex genetic diseases such as cancer, diabetes and cardiovascular disorders (Hughes, T. R. and Shoemaker, D. D. 2001 Curr Opin Chem Biol 5:21-25). This powerful technology reveals disease-specific patterns in gene expression, thereby expediting the identification of candidate genes (Albertson, D. G. and Pinkel, D. 2003 Hum Mol Genet 12:R145-52). To date, there have been only five reports on the application of DNA microarrays to the discovery of endometriosis-related genes. All five studies have compared gene expression profiles of endometriotic tissues obtained during laparoscopy versus normal endometrium, but focusing in different aspects of the disease (i.e., infertility, deep endometriosis, and ovarian endometriomas). In one study by Eyster et al. (2002), eight out of 4,133 genes analyzed were shown to be overexpressed in endometriomas compared with matched uterine endometrium (Eyster, K. M. et al. 2002 Fertil Steril 77:38-42). Overexpressed genes were either cystoskeletal elements (vimentin, β-actin, and α-2-actin), housekeeping genes (40S ribosomal protein S23), or immune related proteins (Igγ light chain, Ig germline H chain, complement, major histocompatibility complex class 1). The authors suggested that the increased level of expression of cytoskeletal proteins such as vimentin could explain the invasiveness potential of endometriotic cells, and that immune cell infiltration in the endometrial implants could account for the observed overexpression of immunoglobulin genes. Arimoto and others (2003) also used DNA microarrays to identify gene-expression profiles of ovarian endometriomas (Arimoto, T. et al. 2003 Int J Oncol 22:551-560). Among the genes shown to be upregulated in endometrial cysts were HLA antigens, complement factors, ribosomal proteins, and transforming growth factor B1 (TGFBI). Genes that were down-regulated included TP53, growth arrest and DNA-damage-inducible proteins (GADD34, GADD45A, and GADD45B), p53-induced protein (PIG11), and oviductal glycoprotein (OVGP1). Lebovic and colleagues (2002) compared gene expression levels of 597 human genes induced by IL-1β in endometriosis versus normal endometrial biopsies (Lebovic, D. I. et al. 2002 Fertil Steril 78:849-854). They observed that the cell-cycle regulatory gene Tob-1 was down-regulated by IL-1β in ectopic stromal cells, and suggested that inhibition in the expression of this gene may promote endometriotic cell growth. Kao et al. (2003) used DNA microarrays to analyze the molecular basis of endometriosis-related infertility (Kao, L. C. et al. 2003 Endocrinology 144:2870-2881). Genes which are normally upregulated in normal endometrium during the window of implantation but were found to be decreased in endometriosis included IL-15 (a strong promoter of NK cell proliferation and function), complement 4 binding protein (C4BP; which may interfere with Wnt signaling by interacting with LRP5), and glycodelin (which is under the regulation of progesterone and has been suggested to interfere with fertilization). Finally, it has recently been shown that deep endometriosis lesions have increased expression of platelet-derived growth factor receptor alpha (PDGFRA), protein kinase C beta 1 (PKCβ1) and janus kinase 1 (JAK1), providing evidence for the involvement of the RAS/RAF IMAPK pathways in the pathophysiology of endometriosis (Matsuzaki, S. et al. 2004 Mol Hum Reprod 10:719-728)

### SEGUE TO THE INVENTION

The objective of the present study was to identify differences in gene expression profiles of blood lymphocytes from endometriosis patients and controls. Although blood may not be the primary target tissue in endometriosis, there is some evidence that this disease is associated with an inflammatory component and may be monitored and even evaluated in peripheral blood lymphocytes (Bedaiwy, M. A. et al. 2002 Hum Reprod 17:426-431). Even if blood lymphocytes are not directly involved in the disease process, gene expression profiles of these cells have been shown to be indicative and diagnostic of disease states (Tang, Y. et al. 2001 Ann Neurol 50:699-707). Thus, we hypothesized that identification of differentially-expressed genes in peripheral blood lymphocytes will reveal gene targets with utility in understanding disease pathogenesis and, very importantly, in the design of specific, non-invasive molecular tests that could facilitate diagnosis. We report the identification of gene targets which are envisioned as forming the basis for diagnosis of endometriosis using blood samples.

### SUMMARY OF THE INVENTION

The invention comprises a method of identifying or predicting the predisposition to endometriosis in a female subject comprising determining the level of gene expression of at least one differentially-expressed gene or protein or peptide of peripheral blood leukocytes in a sample of peripheral blood leukocytes or peripheral blood in a subject to provide a first value, determining the level of gene expression of the at least one differentially-expressed gene or protein or peptide of said leukocytes in a control or reference standard to provide a second value, and comparing whether there is a difference between the first value and second value.

The invention comprises a method where the level of gene expression of a member of the group consisting of LOXL1, IL2RG, LRP5, MPB, TNF, MAN2A2, P4HA1 and PDGF is determined.

The invention also comprises a method where the compared protein or peptide level is increased or decreased for a member of the group consisting of LOXL1, IL2RG, LRP5, MPB, TNF, MAN2A2, P4HA1 and PDGF.

The invention also comprises a method of monitoring a subject identified as having endometriosis before and after treatment comprising determining the level of gene expression of at least one differentially-expressed gene of peripheral blood leukocytes in a sample of peripheral blood leukocytes or peripheral blood in the subject prior to treatment providing a first value, determining the level of gene expression of at least one differentially-expressed gene of said leukocytes after treatment providing a second value, and comparing the difference in the level of gene expression of the subject before treatment and after treatment.

The invention further comprises a method of screening candidate agents for use in treatment of endometriosis comprising contacting a cell capable of expressing at least one differentially-expressed gene with a candidate agent ex vivo, determining the level of gene expression of the at least one differentially-expressed gene in the cell to provide a first value, determining the level of gene expression of the same at least one differentially-expressed gene in a cell in the absence of the candidate agent to provide a second value, and comparing the first value with the second value where a difference in the level of gene expression is indicative of an agent potentially capable of being used for the treatment of endometriosis.

The invention further comprises a method of treating or preventing endometriosis comprising administering to a subject an effective amount of an agent that can induce a decrease or increase in the level of gene expression, synthesis, or activity of at least one differentially-expressed gene or gene expression product.

The invention further comprises a method of manufacture of a medicament for the treatment or prevention of endometriosis comprising an effective amount of an agent that can induce a decrease or increase in the level of gene expression, synthesis, or activity of at least one differentially-expressed gene or gene expression product.

The invention further comprises a kit for identifying or predicting the predisposition to endometriosis in a female subject comprising means for determining the level of gene expression of at least one differentially-expressed gene of peripheral blood leukocytes in a sample of peripheral blood leukocytes or peripheral blood in a subject to provide a first value, determining the level of gene expression of the at least one differentially-expressed gene of said leukocytes in a control or reference standard to provide a second value, and comparing whether there is a difference between the first value and second value.

### BRIEF DESCRIPTION OF THE DRAWINGS

FIG. 1. Expression of the nine most discriminatory genes based on a gene selection program (arrayanalysis.nih.gov, see Example 1). Each row represents a gene and each column represents a sample. For each gene, red (dark gray) color indicates a higher level of expression relative to the mean, green (light gray) indicates a lower level of expression relative to the mean. The scale below indicates the number of standard deviations from the mean. (Clone ID in italics is not an IMAGE clone. UniGene/Gene Symbols are from UniGene build #173).

FIG. 2. Relative gene expression in patients with endometriosis versus normal controls. The y-axis shows the average relative expression (2^{-ΔΔCt}) of the nine most discriminatory genes, after normalization against expression of the housekeeping gene GAPDH. p values were calculated by two-tailed unpaired t tests. **p<0.001; *p<0.01.

FIG. 3. mRNA expression of genes in patients with endometriosis versus normal controls (Set 1).

FIG. 4. mRNA expression of genes in patients with endometriosis versus normal controls (Set 2).

FIG. 5. Relative gene expression in patients with endometriosis versus normal controls (Set 2).

FIG. 6. Relative Expression Comparison Set 1 vs. Set 2.

FIG. 7. Relative Expression (All).

### DETAILED DESCRIPTION OF THE PREFERRED EMBODIMENT

The objective of this study was to identify molecular biomarkers for endometriosis in peripheral blood lymphocytes using DNA microarrays. A case-control study was done as part of a multicenter academic research program. Premenopausal women with or without endometriosis, as determined by OB-GYN specialists during surgery, were analyzed. Microarray analysis included six endometriosis patients and five controls; 15 endometriosis patients and 15 controls were analyzed by real-time RT-PCR. Patients with all disease stages were included. The expression levels of mRNAs in blood lymphocytes from endometriosis patients and controls were compared with those of a standard total RNA. Gene expression data were validated by real-time RT-PCR analysis. A gene selection program identified genes that were differentially-expressed in samples from endometriosis patients. To validate the gene expression data, the nine most discriminatory genes were analyzed by real-time RT-PCR. Two of the nine genes identified, interleukin 2 receptor gamma (severe combined immunodeficiency) (IL2RG), with nucleotide and amino acid sequences, e.g., Genbank Accession Number AY692262 and lysyl oxidase-like 1 (LOXL1) with nucleotide and amino acid sequences, e.g., Genbank Accession Number BC015090, were shown to be significantly differentially-expressed. This is the first report of genes that are differentially-expressed in peripheral blood lymphocytes of patients with endometriosis, which may provide important clues regarding the pathogenesis of this disease. Moreover, they are envisioned as being considered targets for non-invasive diagnostic assays for endometriosis and are contemplated as being validated in a larger population.

Unless defined otherwise, technical and scientific terms used herein have the same meaning as commonly understood by one of ordinary skill in the art to which this invention belongs. See, e.g., Singleton P and Sainsbury D., Dictionary of Microbiology and Molecular Biology 3^{rd} ed., J. Wiley & Sons, Chichester, N.Y., 2001; and The Glossary of Genetics, 5th Ed., R. Rieger et al. (eds.), Springer Verlag (1991).

“Differential expression” in the context of the present invention refers to transcribed expression products and gene expression products (e.g., proteins or peptides, mRNA, cDNA) that are expressed in a different amount in peripheral blood leukocytes of subjects having endometriosis as compared to control subjects (e.g., a person with a negative diagnosis or undetectable endometriosis, normal or healthy subject).

“Transcript” refers to a strand of nucleic acid that has been synthesized using another nucleic acid strand as a template.

“Proteins or polypeptides or peptides” of the present invention are contemplated to include any fragments thereof, in particular, immunologically detectable fragments. One of skill in the art would recognize that proteins which are released by cells could become degraded or cleaved into such fragments. Additionally, certain proteins or polypeptides are synthesized in an inactive form, which may be subsequently activated by proteolysis. Such fragments of a particular protein may be detected as a surrogate for the protein itself.

Proteins may be secreted (exported) or non-secreted. Non-secreted proteins may be intracellular (inside the cell) or on the surface of the cell.

The term “sample” as used herein refers to a sample from a subject obtained for the purpose of identification, diagnosis, prediction, or monitoring. In certain aspects of the invention such a sample may be obtained for the purpose of determining the outcome of an ongoing condition or the effect of a treatment regimen on endometriosis. Preferred test samples include blood, serum, or plasma. In addition, one of skill in the art would realize that some test samples would be more readily analyzed following a fractionation or purification procedure, for example, separation of whole blood into serum or plasma components.

The term “blood” as used herein refers to either whole blood without prior fractionation, peripheral blood leukocytes, peripheral blood mononuclear cells (PBMCs) or another subfraction of blood.

The term “peripheral blood” refers to blood in the systemic circulation.

A difference in the “level of gene expression” or in the “peptide level” is a relative difference. For example, it may be a difference in the level of gene expression of a sample taken from a subject having endometriosis as compared to control subjects or a reference standard. A comparison can be made between the level of gene expression in a subject at risk of endometriosis to a subject known to be free of a given condition, i.e., “normal” or “control”. Alternatively, a comparison can be made to a “reference standard” known to be associated with a good outcome (e.g., the absence of endometriosis) such as an average level found in a population of normal individuals not suffering from endometriosis. According to the present invention, a comparison can be made between the level of gene expression and the identification or predisposition of a subject to develop endometriosis.

The level of gene expression or the level of proteins/peptides present in a sample being tested can be either in absolute amount (e.g., μg/ml) or a relative amount (e.g., relative intensity of signals).

A difference is present between the two samples if the amount of gene expression or the level of proteins/peptides is statistically significantly different from the amount of gene expression or the level of proteins/peptides in the other sample. For example, there is a difference in gene expression or in the level of proteins/peptides between the two samples if the amount of gene expression or the level of proteins/peptides is present in at least about 20%, at least about 30%, at least about 50%, at least about 80%, at least about 100%, at least about 200%, at least about 400%, at least about 600%, at least about 800%, or at least about 1000% greater than it is present in the other sample.

Identifying or predicting the predisposition to endometriosis may be considered as a diagnostic technique. Diagnostic methods differ in their sensitivity and specificity. The skilled artisan often makes a diagnosis, for example, on the basis of one or more diagnostic indicators. In the present invention, these are the expression levels of a differentially-expressed gene, and/or the polypeptide levels thereof. The presence, absence, or amount of the differentially-expressed gene or the polypeptide thereof is indicative of the presence, severity, or absence of the endometriosis.

Multiple determinations of the gene expression of one or more genes and/or of the polypeptide levels can be made as well as determination of a temporal change in gene expression or polypeptide abundancy which can be used to monitor the progress of the disease or a treatment of the disease. For example, gene expression/polypeptide abundancy may be determined at an initial time, and again at a second time. In such aspects, an increase in the gene expression and/or polypeptide level from the initial time to the second time may be diagnostic of endometriosis. Likewise, a decrease in the gene expression and/or polypeptide level from the initial time to the second time may be indicative of a responsiveness of a subject to a particular type of treatment of endometriosis. Furthermore, the change in gene expression of one or more genes may be related to the severity of endometriosis and future adverse events.

In one embodiment of the invention, the level of gene expression of at least one gene is determined and/or the polypeptide level thereof. In one embodiment, the level of gene expression of lysyl oxidase-like 1 (LOXL1), e.g., Genbank Number BC015090; interleukin 2 receptor, gamma (severe combined immunodeficiency) (IL2RG), e.g., Genbank Number AY692262; low density lipoprotein receptor-related protein 5 (LRP5), e.g., Genbank Number AF064548; myelin basic protein (MBP), e.g., Genbank Number L18866; tumor necrosis factor (TNF superfamily, member 2; TNF), e.g., Genbank Number BC028148; mannosidase, alpha, class 2A, member 2 (MAN2A2), e.g., Genbank Number NM_{—}006122; procollagen-proline, 2-oxoglutarate 4-dioxygenase (proline 4-hydroxylase) alpha polypeptide 1 (P4HA1), e.g., Genbank Number AK222960; or platelet derived growth factor D/DNA-damage inducible protein 1 (PDGFD), e.g., Genbank Number AF336376, is determined and/or the polypeptide level thereof. In another embodiment, the level of gene expression of a plurality of LOXL1, IL2RG, LRP5, MPB, TNF, MAN2A2, P4HA1 and PDGF are determined.

The skilled artisan will understand that, while in certain aspects comparative measurements of gene expression are made of the same gene at multiple time points, one could also measure a given gene at one time point, and a second gene at a second time point, and a comparison of the gene expression of these genes may provide diagnostic information or monitor the progress of the disease.

In one aspect of the invention, gene expression of one or more genes may be comparatively measured at different time points.

The phrase “probability of the presence of endometriosis” as used herein refers to methods by which the skilled artisan can predict the condition in a subject. It does not refer to the ability to predict the endometriosis with 100% accuracy. Instead, the skilled artisan will understand that it refers to an increased probability that endometriosis is present or will develop. For example, endometriosis is more likely to occur in a subject having high levels of expression of IL2RG and/or increased levels of IL2RG polypeptide and/or decreased levels of expression of LOXL1 and/or decreased levels of LOXL1 polypeptide when compared to a control or reference standard such as a subject not being affected by or having a predisposition to endometriosis. In one aspect of the invention, the probability of the presence of endometriosis is about a 50% chance, about a 60% chance, about a 75% chance, about a 90% chance, and about a 95% chance. The term “about” in this context refers to ±1%.

The skilled artisan will understand that associating a particular gene with a predisposition to endometriosis is a statistical analysis. Additionally, a change in gene expression and/or peptide level from baseline levels may be reflective of patient prognosis, and the degree of change in gene expression may be related to the severity of adverse events. Statistical significance is often determined by comparing two or more populations, and determining a p value. Preferred p values are 0.1, 0.05, 0.025, 0.02, 0.01, 0.005, 0.001, and 0.0001.

In a further aspect, the invention relates to kits for identification of endometriosis in a subject. These kits comprise devices and reagents for measuring gene expression and/or determining polypeptide levels in a subject's sample and instructions for performing the assay and interpreting the results. Such kits preferably contain sufficient reagents to perform one or more such determinations.

The “sensitivity” of an assay according to the present invention is the percentage of diseased individuals (those with endometriosis) who test positive (percent of “true positives”). Diseased individuals not detected by the assay are “false negatives”. Subjects who are not diseased and who test negative in the assay, are termed “true negatives.” The “specificity” of a diagnostic assay is 1 minus the false positive rate, where the “false positive rate” is defined as the proportion of those without the disease who test positive. While a particular diagnostic method may not provide a definitive diagnosis of a condition, it suffices if the method provides a positive indication that aids in diagnosis.

### Measurement of Gene Expression

Numerous methods and devices are well known to the skilled artisan for the detection and analysis of the gene expression and measurement of polypeptide levels of the present invention. The term “gene expression” refers to the presence or amount of a specific gene including, but not limited to, mRNA, cDNA or the polypeptide, peptide or protein expression product of a specific gene. In a preferred aspect of the invention, the gene expression of LOXL1, IL2RG, LRP5, MPB, TNF, MAN2A2, P4HA1 and/or PDGF and/or the level of corresponding polypeptides are determined.

In one embodiment of the invention, the gene expression is determined by measuring RNA levels. Gene expression may be detected using a PCR-based assay. For example, reverse-transcriptase PCR (RT-PCR) is used to detect the expression of RNA. In RT-PCR, RNA is enzymatically converted to cDNA using a reverse-transcriptase enzyme. The cDNA is then used as a template for a PCR reaction. PCR products can be detected by any suitable method including, but not limited to, gel electrophoresis and staining with a DNA-specific stain or hybridization to a labeled probe. In yet another aspect of the invention, the quantitative RT-PCR with standardized mixtures of competitive templates can be utilized.

In another embodiment of the present invention, gene expression is detected using a hybridization assay. In a hybridization assay, the presence or absence of biomarker is determined based on the ability of the nucleic acid from the sample to hybridize to a complementary nucleic acid molecule, e.g., an oligonucleotide probe. A variety of hybridization assays are available. In some embodiments of the invention, hybridization of a probe to the sequence of interest is detected directly by visualizing a bound probe, e.g., a Northern or Southern assay. In these assays, DNA (Southern) or RNA (Northern) is isolated. The DNA or RNA is then cleaved with a series of restriction enzymes that cleave infrequently in the genome and not near any of the markers being assayed. The DNA or RNA is then separated, e.g., on an agarose gel, and transferred to a membrane. A labeled probe or probes, e.g., by incorporating a radionucleotide, is allowed to contact the membrane under low-, medium- or high-stringency conditions. Unbound probe is removed and the presence of binding is detected by visualizing the labeled probe. In the present invention, the gene expression is determined for LOXL1, IL2RG, LRP5, MPB, TNF, MAN2A2, P4HA1 and/or PDGF.

### Nucleic Acid Arrays

A nucleic acid array comprises any combination of the nucleic acid sequences generated from, or complementary to nucleic acid transcripts, or regions thereof, including the species of nucleic acid transcripts present in blood. A microarray according to the invention preferably comprises between 10, 100, 500, 1000, 5000, 10,000 and 15,000 nucleic acid members, and more preferably comprises at least 5000 nucleic acid members. The nucleic acid members are known or novel nucleic acid sequences described herein, or any combination thereof. A microarray according to the invention is used to assay for differential levels of species of transcripts RNA expression profiles present in blood samples from healthy patients as compared to patients with a disease.

Microarrays include those arrays which encompass transcripts that are expressed in an individual. In one embodiment, a microarray encompasses transcripts that are expressed in humans. In another embodiment, microarrays of the invention can be either cDNA based arrays or oligonucleotide based arrays.

### Quantitative Real Time RT-PCR

In another aspect of the invention, the level of one or more species of transcripts of the invention can be determined using quantitative methods including QRT-PCR, RNA from blood using quantitative reverse transcription (RT) in combination with the polymerase chain reaction (PCR).

Total RNA, or mRNA from blood is used as a template and a primer specific to the transcribed portion of a gene of the invention is used to initiate reverse transcription. Primer design can be accomplished utilizing commercially available software (e.g., Primer Designer 1.0, Scientific Sofware etc.). The product of the reverse transcription is subsequently used as a template for PCR.

PCR provides a method for rapidly amplifying a particular nucleic acid sequence by using multiple cycles of DNA replication catalyzed by a thermostable, DNA-dependent DNA polymerase to amplify the target sequence of interest. PCR requires the presence of a nucleic acid to be amplified, two single-stranded oligonucleotide primers flanking the sequence to be amplified, a DNA polymerase, deoxyribonucleoside triphosphates, a buffer and salts.

The method of PCR is well known in the art. PCR, is performed as described in Mullis and Faloona, 1987, Methods Enzymol., 155:335.

PCR is performed using template DNA or cDNA (at least fg; more usefully, 1-1000 ng) and at least 25 pmol of oligonucleotide primers. A typical reaction mixture includes: 100 ng of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is optionally overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

QRT-PCR which is quantitative in nature can also be performed, using either reverse transcription and PCR in a two step procedure, or reverse transcription combined with PCR in a single step protocol so as to provide a quantitative measure of the level of one or more species of RNA transcripts in blood. One of these techniques, for which there are commercially available kits such as Taqman®) (Perkin Elmer, Foster City, Calif.), is performed with a transcript-specific antisense probe. This probe is specific for the PCR product (e.g., a nucleic acid fragment derived from a gene) and is prepared with a quencher and fluorescent reporter probe complexed to the 5′ end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5′-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96 well plates so that samples derived from many individuals are processed and measured simultaneously. The Taqman system has the additional advantage of not requiring gel electrophoresis and allows for quantification when used with a standard curve.

A second technique useful for detecting PCR products quantitatively without electrophoresis is to use an intercalating dye such as the commercially available QuantiTect™ SYBR® Green PCR (Qiagen, Valencia Calif.). RT-PCR is performed using SYBR® green as a fluorescent label which is incorporated into the PCR product during the PCR stage and produces a fluorescence proportional to the amount of PCR product.

Both Taqman® and QuantiTect™ SYBR® systems can be used subsequent to reverse transcription of RNA.

Additionally, other systems to quantitatively measure the level of one or more species of transcripts are known including Molecular Beacons which uses a probe having a fluorescent molecule and a quencher molecule, the probe capable of forming a hairpin structure such that when in the hairpin form, the fluorescence molecule is quenched, and when hybridized the fluorescence increases giving a quantitative measurement of one or more species of RNA transcripts.

Several other techniques for detecting PCR products quantitatively without electrophoresis may also be used according to the invention (see for example PCR Protocols, A Guide to Methods and Applications, Innis et al., Academic Press, Inc. N.Y., (1990)).

### Measurement of Protein Expression

In yet another embodiment of the invention, the gene expression is determined by measuring polypeptide gene expression products. In a preferred aspect of the invention, gene expression is measured by identifying the amount of one or more polypeptides encoded by the genes for LOXL1, IL2RG, LRP5, MPB, TNF, MAN2A2, P4HA1 and/or PDGF. The present invention is not limited by the method in which gene expression is detected or measured.

A protein or polypeptide or peptide expression product encoded by the genes for LOXL1, IL2RG, LRP5, MPB, TNF, MAN2A2, P4HA1 and/or PDGF may be detected by a suitable method. With regard to peptides, polypeptides or proteins in samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

The presence or amount of a protein or polypeptide or peptide is generally determined using specific antibodies and detecting specific binding. Any suitable immunoassay may be utilized, for example, enzyme-linked immunoassays (ELISA), radioimmunoassays (RIAs), competitive binding assays, and the like. Specific immunological binding of the antibody to the protein or polypeptide can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the proteins or polypeptides are also contemplated by the present invention. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on solid support. This strip can then be dipped into the test sample and then processed quickly through washes and detection steps to generate a measurable signal, such as a colored spot.

The analysis of a plurality of genes and/or polypeptides of the present invention may be carried out separately or simultaneously with one test sample. In addition, one skilled in the art would recognize the value of testing multiple samples (for example, at successive time points) from the same individual. Such testing of serial samples allows the identification of changes in gene expression and/or polypeptide levels over time. Increases or decreases in gene expression levels, as well as the absence of change in gene expression and/or polypeptide levels, can provide useful information about the disease status that includes, but is not limited to identifying the approximate time from onset of the event, the presence and amount of salvageable sample, the appropriateness of drug therapies, the effectiveness of various therapies as indicated by resolution of symptoms, differentiation of the various types of endometriosis, identification of the severity of the event, identification of the disease severity, and identification of the patient's outcome, including risk of future events.

A panel comprising of the genes referenced above may be constructed to provide relevant information related to the diagnosis or prognosis of endometriosis and management of subjects with endometriosis. Such a panel can be constructed preferably using the sequences of LOXL1, IL2RG, LRP5, MPB, TNF, MAN2A2, P4HA1 and/or PDGF. The analysis of a single gene or subsets of genes comprising a larger panel of genes alone or in combination with the analysis of a single polypeptide or a subset of polypeptides can be carried out by one skilled in the art to optimize sensitivity or specificity.

The analysis of gene expression and/or determination of polypeptide levels can be carried out in a variety of physical formats as well. For example, the use of microtiter plates or automation could be used to facilitate the processing of large numbers of test samples in a high throughput manner.

In another aspect of the invention, an array is provided to which probes that correspond in sequence to gene products, e.g., cDNAs, mRNAs, cRNAs, polypeptides and fragments thereof, can be specifically hybridized or bound at a known position. In one embodiment of the invention, the array is a matrix in which each position represents a discrete binding site for a product encoded by a gene for LOXL1, IL2RG, LRP5, MPB, TNF, MAN2A2 and/or PDGF. In another aspect of the invention, the “binding site”, hereinafter “site”, is a nucleic acid or nucleic acid analogue to which a particular cognate cDNA can specifically hybridize. The nucleic acid or analogue of the binding site can be, e.g., a synthetic oligomer, a fall-length cDNA, a less than full-length cDNA or a gene fragment.

In another aspect, the present invention provides a kit for the analysis of gene expression and/or polypeptide levels. Such a kit preferably comprises devices and reagents for the analysis of at least one test sample and instructions for performing the assay. Optionally the kits may contain one or more means for converting gene expression and/or amounts of polypeptides to a diagnosis or prognosis of endometriosis in a subject. Comparison of the subject's gene expression pattern, with the controls or reference standards, would indicate whether the subject has endometriosis.

In one embodiment of the invention, the kits contain antibodies specific for at least one of LOXL1, IL2RG, LRP5, MPB, TNF, MAN2A2, P4HA1 and/or PDGF. In other embodiments, the kits contain reagents specific for the detection of nucleic acid, e.g., oligonucleotide probes or primers. In some embodiments, the kits contain all of the components necessary to perform a detection assay, including all controls and instructions for performing assays and for analysis of results. In one embodiment of the invention, the kits contain instructions including a statement of intended use as required by the U.S. Food and Drug Administration (FDA) or foreign counterpart for the labeling of in vitro diagnostic assays and/or of pharmaceutical or food products.

In another aspect of the present invention, a method of screening agents for use in the treatment of endometriosis is provided. In particular, agents that can induce a decrease in the level of gene expression, synthesis or activity of IL2RG and/or induce an increase in the level of gene expression, synthesis or activity of LOXL1 are contemplated.

For example, in one embodiment one would first treat a test subject known to have endometriosis with a test agent and then analyze a representative sample of the subject for the level of expression of the genes or sequences which change in expression in response to endometriosis and/or for the level of polypeptide. One then compares the analysis of the sample with a control known to have endometriosis but not given the test compound and thereby identifies test compounds that are capable of modifying the gene expression.

In another embodiment of the present invention, one would base a therapy on the sequences of LOXL1, IL2RG, LRP5, MPB, TNF, MAN2A2, P4HA1 and/or PDGF. For example, one would try to decrease the expression of IL2RG and to induce an increase in the level of LOXL1.

Methods of increasing or decreasing the expression of said genes would be known to one of skill in the art. Examples for supplementation of expression would include supplying subject with additional copies of the gene. One example for decreasing expression would include RNA antisense technologies or pharmaceutical intervention.

### Identification of Molecular Markers for Endometriosis in Blood Lymphocytes Using DNA Microarrays

### Microarray Data Analysis

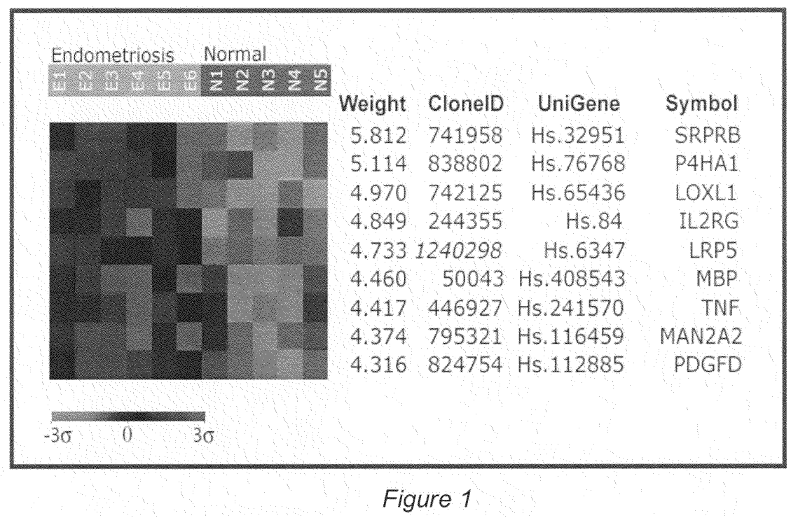

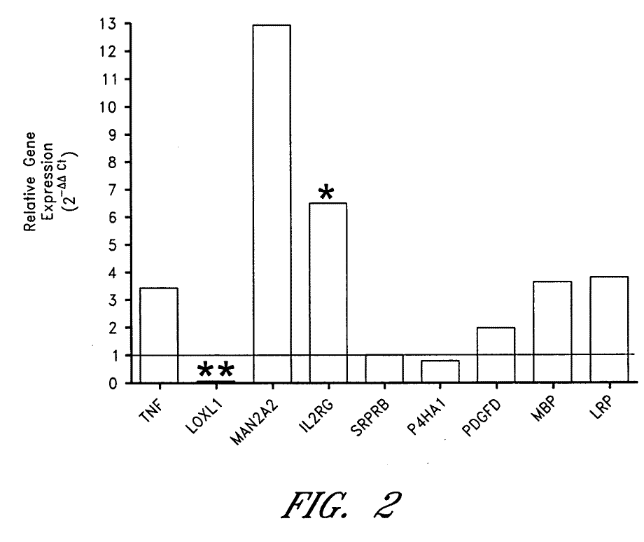

Data were analyzed as described in Example 1. The fold-difference considered as overexpression was set at ≧2, as generally used in DNA microarray data analysis. We analyzed a total of 15,097 cDNA clones (14,185 known genes and 912 expressed sequence tag sites) using peripheral blood lymphocyte total RNA from patients and controls. Each gene's discriminative capacity to separate the two groups was assessed by t-statistics (weight, see Table 1). The most discriminatory genes, both up- and down-regulated, were listed. From those genes found to be overexpressed in patients versus controls, we selected nine which had a weight greater than 4 and a mean ratio difference greater than 2.0 for further analysis (FIG. 1). These were: the signal recognition particle receptor, B subunit (SRPRB); procollagen-proline, 2-oxoglutarate 4-dioxygenase (proline 4-hydroxylase) alpha polypeptide 1 (P4HA1); lysyl oxidase-like 1 (LOXL1); interleukin 2 receptor, gamma (severe combined immunodeficiency) (IL2RG); low density lipoprotein receptor-related protein 5 (LRP5); myelin basic protein (MBP); tumor necrosis factor (TNF superfamily, member 2; TNF); mannosidase, alpha, class 2A, member 2 (MAN2A2); and platelet derived growth factor D/DNA-damage inducible protein 1 (PDGFD) (Table 1). The expression of all nine genes was ≧2-fold increased in peripheral blood lymphocytes of patients as compared to controls.

TABLE 1
Microarray and relative real-time RT-PCR analysis
of genes in endometriosis patients and controls.

| | Microarray | | | |
|---|---|---|---|---|
| | | Weight | RT-PCR | |
| | Fold- | Microarray | Fold- | P |
| Gene | expression | analysis^{a} | expression^{b} | RT-PCR |
| SRPRB | 3.59 | 5.812 | 1.02 | 0.2369 |
| P4HA1 | 3.74 | 5.114 | 0.77 | 0.0594 |
| LOXL1 | 3.96 | 4.970 | 0.06 | 0.0002 |
| IL2RG | 2.32 | 4.849 | 6.49 | 0.0037 |
| LRP5 | 2.92 | 4.733 | 3.80 | 0.1274 |
| MBP | 3.03 | 4.460 | 3.62 | 0.6503 |
| TNF | 2.79 | 4.417 | 3.44 | 0.2973 |
| MAN2A2 | 2.41 | 4.374 | 12.93 | 0.1998 |
| PDGFD | 2.56 | 4.316 | 1.96 | 0.6708 |

^{a}Results shown are for six patients vs. five controls.
^{b}Results shown are for 15 patients vs. 15 controls. Discriminative weight values were determined as described in Example 1. p-values for the real-time RT-PCR data were determined using two-tailed unpaired t tests, significance was set at 0.05.

### Validation of Microarray Data Using Real-Time RT-PCR

The expression of the nine most discriminatory genes was further evaluated in blood lymphocytes from additional patients (N=15) and controls (N=15) using relative real-time RT-PCR. The results are summarized in Table 1. Two of the nine genes identified were confirmed as differentially expressed by real-time RT-PCR: IL2RG was upregulated (6.49-fold; p=0.0037) and LOXLI was downregulated (0.06-fold; p=0.0002) in patients versus controls (FIG. 2). Although LRP5, MBP, TNF, MAN2A2, and PDGFD were upregulated over 2-fold as shown by RT-PCR, the difference in gene expression levels between cases and controls did not reach statistical significance.

### Discussion

In the past years the number of studies devoted to finding biomarkers has greatly increased (Colburn, W. A. 2003 J Clin Pharmacol 43:329-341; Frank, R. and Hargreaves, R. 2003 Nat Rev Drug Discov 2:566-580). According to the Biomarkers Definitions Working Group, a biomarker is “a characteristic that is objectively measured and evaluated as an indicator of normal biological processes, pathogenic processes, or pharmacologic responses to a therapeutic intervention” (Biomarkers Definitions Working Group 2001 Clin Pharmacol Ther 69:89-95). However, the number of potential diagnostic biomarkers is much smaller than those considered potential targets for drug development (Levenson, V. V. 2004 Pharmacogenomics 5:459-461). Global analysis of gene expression might accelerate the finding of new diagnostic or prognostic biomarkers that in turn would need to be validated in a broader population.

Hence, we undertook this study with the premise that characterization of the pattern of gene expression in peripheral blood lymphocytes, a very accessible tissue, may expedite the identification of genes that could serve as diagnostic or prognostic biomarkers for endometriosis. We hypothesized that microarray analysis of the transcript profile of blood lymphocytes could better serve the purpose of finding a non-invasive marker for this disease, as opposed to studying endometriosis implants, endometriomas, or peritoneal fluid. We based this hypothesis on recent studies showing significant differences in the levels of various inflammatory molecules/growth factors in both serum and peritoneal fluid of women with versus without endometriosis (Bedaiwy, M. A. et al. 2002 Hum Reprod 17:426-431; Navarro, J. 2003 Obstet Gynecol Clin North Am 30:181-192; Iwabe, T. et al. 2002 Gynecol Obstet Invest 53 Suppl 1:19-25). Also, there is ample evidence for genetic differences between patients with endometriosis and unaffected individuals, and such differences could be evident in blood lymphocytes (Taylor, R. N. et al. 2002 Fertil Steril 78:694-698; Nakago, S. et al. 2001 Mol Hum Reprod 7:1079-1083). Finally, it is widely accepted that the immune system plays an important role in endometriosis, and thus, analysis of systemic immune responses could reflect the local (i.e., peritoneal cavity) ones (Gagne, D. et al. 2003 Fertil Steril 80:43-53; Gagne, D. et al. 2003 Fertil Steril 80:876-885).

Thus far, only a few potential markers for endometriosis-mainly cytokines, growth factors, adhesion molecules, and hormones have been detected in serum or blood lymphocytes. Bedaiwy and collaborators (2002) showed that serum IL-6 and peritoneal fluid TNF-a could be used to discriminate between patients with and without endometriosis with a high degree of sensitivity and specificity (Bedaiwy, M. A. et al. 2002 Hum Reprod 17:426-431; Bedaiwy, M. A. and Falcone, T. 2004 Clin Chim Acta 340:41-56). However, measurement of peritoneal fluid TNF-α would still require an invasive procedure and therefore could not constitute the basis of a simplified test for endometriosis. Barrier and Sharpe-Timms (2002) reported that women with advanced stage endometriosis had higher serum levels of VCAM-1 and lower serum levels of ICAM-1 (Barrier, B. F. and Sharpe-Timms, K. L. 2002 J Soc Gynecol Investig 9:98-101). They concluded that aberrant levels of soluble adhesion molecules not only help explain the pathogenesis of endometriosis, but can also be used as biochemical markers for staging the disease. Pizzo and coworkers (2002) showed that patients with endometriosis had higher serum levels of TNF-α, IL-8 and MCP-1, all of which decreased with disease severity-while serum TGF-β levels, on the other hand, increased with severity (Pizzo, A. et al. 2002 Gynecol Obstet Invest 54:82-87). In addition, soluble levels of vascular endothelial growth factor (VEGF) were also significantly increased in patients as compared to controls, although this observation could not be replicated by another study (Matalliotakis, I. M. et al. 2004 Int Immunopharmacol 4:159-160; Gagne, D. et al. 2003 Hum Reprod 18:1674-1680). Other proteins that are significantly increased in the serum of endometriosis patients are luteinizing hormone (LH), Fas ligand, soluble tumor necrosis factor receptors, and ICAM-1-although the latter two were significantly higher in patients only in a particular phase of the menstrual cycle (Illera, J. C. et al. 2001 Reproduction 121:761-769; García-Velasco, J. A. et al. 2002 Fertil Steril 78:855-859; Koga, K. et al. 2000 Mol Hum Reprod 6:929-933; Steff, A. M. et al. 2004 Hum Reprod 19:172-178). Taken together, all of the above studies have failed to identify non-invasive markers of endometriosis which could be useful to diagnose patients of all disease stages and whose expression does not depend on the menstrual cycle phase of the patient at the time of the test.

To date, there are only five reports on the use of the DNA microarray technology to identify endometriosis-specific patterns of gene expression (Hughes, T. R. and Shoemaker, D. D. 2001 Curr Opin Chem Biol 5:21-25; Albertson, D. G. and Pinkel, D. 2003 Hum Mol Genet 12:R145-52; Eyster, K. M. et al. 2002 Fertil Steril 77:38-42; Arimoto, T. et al. 2003 Int J Oncol 22:551-560; Lebovic, D. I. et al. 2002 Fertil Steril 78:849-854; Kao, L. C. et al. 2003 Endocrinology 144:2870-2881; Matsuzaki, S. et al. 2004 Mol Hum Reprod 10:719-728; Giudice, L. C. et al. 2002 Ann NY Acad Sci 955:252-264; Discussion 293-295, 396-406). However, none of these studies have specifically addressed the fact that there are no specific serum- or blood-based diagnostic tests for this debilitating disease. Therefore, though important data has been collected which will certainly help dissect the molecular mechanisms involved in the pathophysiology of endometriosis, these studies have failed to identify a common specific marker that can facilitate diagnosis and treatment monitoring by testing blood or sera. Moreover, since these studies have looked at different disease subtypes (i.e., infertility vs. deep endometriosis vs. ovarian endometriosis), which could in fact be characterized by distinct gene expression profiles, the reported data is divergent and can not be broadly applied.

In order to identify molecular biomarkers for endometriosis in blood, we compared gene expression of ˜15,000 genes/ESTs in blood lymphocytes isolated from six patients and five controls. Up- and down-regulated genes were listed, and each gene's discriminative capacity to separate the two study groups was assessed by t-statistics as described in Example 1. Among the overexpressed genes, we further analyzed the nine with a weight greater than 4.0 and a group-wise averaged fold change greater than 2. These were classified in the following groups: i) proteins involved in the immune response: TNF, a pro-inflammatory cytokine previously shown to be present at increased levels in the peritoneal fluid of endometriosis patients (Eisermann, J. et al. 1988 Fertil Steril 50:573-579), and the IL-2 R γ chain (IL2RG), also known as common gamma chain, an important component of functional IL-2, IL-4, and IL-7 receptors (Nakajima, H. et al. 1997 Exp Med 185:189-195); ii) enzymes involved in collagen metabolism: prolyl-4 hydroxylase (P4HA1) catalyzes the formation of 4-hydroxyproline in collagens (Annunen, P. et al. 1997 J Biol Chem 272:17342-17348), and lysyl oxidase-like 1 (LOXL1) mediates the formation of insoluble collagen in the extracellular matrix (Molnar, J. et al. 2003 Biochim Biophys Acta 1647:220-224); iii) genes involved in promotion of cell proliferation/tumorigenesis: α-mannosidase (MAN2A2), a glycosyl hydrolase that processes N-linked glycans and whose expression level has been correlated with malignant behavior in vitro (Misago, M. et al. 1995 Proc Natl Acad Sci USA 92:11766-11770; Yue, W. et al. 2004 Int J Cancer 108:189-195); the low density lipoprotein receptor 5 (LRP5), which functions as co-receptor of the oncogene Wnt, and has been shown to positively regulate cell proliferation and invasiveness of breast cancer cells (Li, Y. et al. 1998 Invasion Metastasis 18:240-251); the signal recognition particle B subunit (SRPRB), which is upregulated in apoptotic and malignant cells (Yan, W. et al. 2003 World J Gastroenterol 9:1719-1724); and platelet derived growth factor D/DNA-damage inducible protein 1 (PDGFD) characterized by its mitogenic effect in cells of mesenchymal origin (Hamada, T. et al. 2000 FEBS Lett 475:97-102); and iv) myelin basic protein (MBP), the major protein of myelin sheath, which is expressed in the central nervous system and in hematopoietic cells, and the expression of which has been shown to be increased by TNF (Malty, M. C. et al. 2002 Proc Natl Acad Sci USA 99:8856-8861; Huang, C. J. et al. 2002 Int J Dev Neurosci 20:289-296). When additional samples from patients and controls were analyzed using real-time R T-PCR, the differences in relative mRNA expression of IL2RG and LOXL 1 were shown to be significant.

IL-2RG, or common gamma chain, is part of all known T cell growth factor receptors (e.g., IL-2, IL-4, IL-7, IL-9, IL-15, and IL-21) (Habib, T. et al. 2003 J Allergy Clin Immunol 112:1033-1045). As such, this receptor chain is critically involved in the generation of signals that mediate the development of Thl immune responses. It is well known that endometriosis patients show increased levels of activated immune cells and soluble cytokines including IL-2 and IL-4 (Iwabe, T. et al. 2002 Gynecol Obstet Invest 53 Suppl 1:19-25; Szyllo, K. et al. 2003 Mediators Inflamm 12:131-138; Wu, M. Y. and Ho, H. N. 2003 Am J Reprod Immunol 49:285-296). Moreover, baboons with stage II to IV endometriosis have been shown to have increased levels of IL-2R⁺ cells in peripheral blood (D'Hooge, T. M. et al. 1996 Human Reprod 11:1736-1740). The expression of this gene is seen to be increased in patients with endometriosis. LOXL1, on the other hand, has been implicated as a tumor suppressor gene, although this role has not been fully elucidated (Contente, S. et al. 1990 Science 249:796-798; Hamalainen, E. R. et al. 1995 J Biol Chem 270:21590-21593). LOXLI is involved in the TGF-beta signal transduction pathway, and has been shown to be downregulated in head and neck squamous cell carcinoma and prostate cancer (Dairkee, S. H. et al. 2004 BMC Genomics 5:47; Rost, T. et al. 2003 Anticancer Res 23:1565-1573; Ren, C. et al. 1998 Cancer Res 58:1285-1290). Although we can not explain the discrepancies between the real-time RT-PCR and microarray data for LOXL1, the fact that this putative tumor suppressor gene is dramatically downregulated in all the endometriosis patients studied using RT-PCR is intriguing and deserves further investigation. Discrepancies between microarray and real-time PCR results in both direction and level of expression have been reported before (Orr, W. E. et al. 2003 Mol Vis 9:482-496; Jenson, S. D. et al. 2003 Mol Pathol 56:307-312; Ginestier, C. et al. 2002 Am J Pathol 161:1223-1233; Mutch, D. M. et al. November 2001 Genome Biol 2:PREPRINT0009 (electronic publication); Goodsaid, F. M. et al. 2004 Environ Health Perspect 112:456-460). Since RT-PCR is considered the gold standard in gene expression level determination (Goodsaid, F. M. et al. 2004 Environ Health Perspect 112:456-460), and in view of the fact that LOXL1 was dramatically reduced in all of the patients analyzed by RT-PCR, the latter results possibly better reflect what is going on in the patient.

RT-PCR analysis of additional samples was able to confirm the microarray data for LRP5, MBP, TNF, MAN2A2, and PDGFD, although the differences in relative mRNA expression between patients and controls did not reach statistical significance. This may be due to individual differences and the small sample size analyzed. Although the expression of MAN2A2 was 12.93-fold higher in patients than in controls, a large standard error (7.4) might explain the fact that this difference did not reach statistical significance. Of note, TNF has been strongly implicated in the pathophysiology of endometriosis. Its expression, however, has been shown to vary according to menstrual cycle phase (Hunt, J. S. et al. 1997 J Reprod Immunol 35:87-99), and higher levels have been demonstrated in patients with mild versus severe disease (Pizzo, A. et al. 2002 Gynecol Obstet Invest 54:82-87), which can explain the lack of significance for this particular gene. The present study also showed that the expression of PDGFD was upregulated in patient's samples as shown by DNA microarray analysis. This observation together with data reported by Matsuzaki et al. (2004) support a key role for the platelet-derived growth factor system in this disease: they reported the increased expression of the PDGF receptor in deep endometriosis lesions, while we demonstrate that its ligand is upregulated in blood lymphocytes of patients with endometriosis. PDGFD has been shown to stimulate cell proliferation and transformation, and to play a role in angiogenesis (Ustach, C. V. et al. 2004 Cancer Res 64:1722-1729; Li, H. et al. 2003 Oncogene 22:1501-1510); therefore, it is tempting to speculate that activation of the PDGF system may help promote the growth of endometrial cells at ectopic sites.

This is the first report of genes that are differentially-expressed in peripheral blood lymphocytes of patients with endometriosis, which may provide important clues regarding the pathogenesis of this disease. Our analyses have lead to the identification of genes whose segregation with disease, structural alterations, or expression profiles are contemplated as being further studied, in order to better define their use as markers of disease. In another embodiment, we propose that analysis of gene expression levels of a combination of genes would increase the specificity and sensitivity of a minimally invasive detection method for endometriosis. Also, validation studies conducted in a larger population by microarray analysis and RT-PCR are envisioned as being complemented by measurements of serum protein levels of the candidate genes and by high-density tissue microarray analysis of endometriosis samples.

The patients that were analyzed in the present study could be grouped in various different clinical categories according to disease severity (e.g., severe; moderate; mild; minimal) or symptoms (e.g., infertility vs. dysmenorrhea vs. both); this analysis, in turn, might reflect different genetic classes. Also, it would be important to consider factors which may affect lymphocyte gene expression patterns, such as menstrual cycle phase, concurrent infections, and current medications (Willis, C. et al. 2003 Hum Reprod 18:1173-1178; Dosiou, C. et al. 2004 J Clin Endocrinol Metab 89:2501-2504). Because of the small number of patients in each category it was not possible to compare the difference among these classes. Therefore, based on the present data, expression analysis comparing the endometriosis patients with the controls was only able to detect differences in gene expression that are common to all the patient categories studied. Herein lays the value of these findings-differences in gene expression for IL-2RG and LOXLI were significant regardless of disease subtype, current medications or patient menstrual cycle stage. Ongoing prospective studies at our laboratories are specifically designed to elucidate the pattern of expression of the genes of interest, i.e., whether differences in gene expression levels vary according to the menstrual cycle phase, and whether such differences can be related to clinical presentation or disease characteristics (e.g., severe vs. mild disease; dysmenorrhea vs. infertility).

In summary, this study has revealed new target genetic loci in endometriosis, which are envisioned as serving as the basis for the development of non-invasive, specific diagnostic assays as well as novel therapies for this chronic, debilitating disease.

### Example 1

### Study Population

Study subjects (patients and controls) were recruited by direct referrals from collaborating OB-GYNs practicing throughout Puerto Rico. The patient population under study consisted of premenopausal women who had been diagnosed with endometriosis by an OB-GYN specialist during surgery, and included patients with all stages of disease: severe (11), moderate (6), mild (3), minimal (1). Patient samples used for the microarrays (N=6; age range 32-39 y/o; average=35.5 y/o) and real-time RT-PCR validation experiments (N=15; age range 26-39 y/o; average=31.2 y/o) were selected randomly from our nucleic acid bank. Thirteen out of 21 patients (62%) were not taking any medications when the samples were obtained. Those on medications were being treated with GnRH agonists (6), oral contraceptives (1), and danocrine (1). Controls (N=4 for microarrays; N=15 for RT-PCR) were women who underwent laparoscopy or laparotomy for unrelated gynecologic conditions (e.g., uterine fibroids, DUB, sterilization), and who did not have endometriosis as confirmed by surgery. A sample obtained from a male volunteer was included as a control in the microarray experiments. Control samples were completely anonymous and therefore not linked to demographic information. Samples from patients and controls used for the microarrays and the RT-PCR experiments did not overlap. Prior to any experimentation, this research protocol was evaluated and approved by the IRB Committees of both Ponce School of Medicine and NHGRI-NIH. All participants read and signed an informed consent form prior to their entry into the study.

### Blood Samples

After written informed consent was obtained, blood samples were collected by venipuncture by a research nurse, using standard aseptic procedures. Once in the laboratory, lymphocytes were first isolated from whole blood by centrifugation at 2000 rpm for 40 minutes in Histopaque (Sigma, St. Louis, Mo.). Total RNA was isolated from the lymphocytes using Trizol LS and following the manufacturer's specifications (Invitrogen, Carlsbad, Calif.). Expression analysis by cDNA microarrays: We analyzed six blood samples of affected women and five controls using a gene selection program (see below). The arrays used had 15097 cDNA clones that were prepared and printed on glass slides as previously described (DeRisi, J. et al. 1996 Nat Genet 14:457-460; Shalon, D. et al. 1996 Genome Res 6:639-645; Mousses, S. et al. in Gene expression analysis by cDNA microarrays, in Functional Genomics, F. J. Livesey and S. P. Hunt (Eds.), 2000, Oxford University Press, Oxford, pp. 113-137). Of these clones, 912 were expressed sequence tags and the 14,185 remaining clones were known genes. Hybridization and post-hybridization washes were carried out as described before (Mousses, S. et al. in Gene expression analysis by cDNA microarrays, in Functional Genomics, F. J. Livesey and S. P. Hunt (Eds.), 2000, Oxford University Press, Oxford, pp. 113-137; Monni, O. et al. 2001 Proc Natl Acad Sci USA 98:5711-5716; Pollack, J. R. et al. 2002 Proc Natl Acad Sci USA 99:12963-12968). Briefly, approximately 15 to 20 μg of total RNA from subjects' blood lymphocytes and same amount of a standard reference (Universal Human Reference RNA, Stratagene, La Jolla, Calif.) were labeled by reverse transcription using SuperScript Reverse Transcriptase (Invitrogen, Carlsbad, Calif.), and with either Cy3-dUTP or Cy5-dUTP (Amersham-Pharmacia, Piscataway, N.J.), respectively. The labeled probes were subjected to alkaline hydrolysis, purified and concentrated using Microcon 30 (Millipore, Billerica, Calif.). Hybridizations were performed overnight (16-24 hr) in an aqueous solution at 65° C. in a sealed humidified chamber.

Statistical Analysis of cDNA Microarray Data

To identify genes with significant differential expression between the two groups (with and without endometriosis), a gene selection program (arrayanalysis.nih.gov) was employed as previously described (Bittner, M. et al. 2000 Nature 406:536-540; Hedenfalk, I. et al. 2001 N Engl J Med 344:539-548). Gene expression data were first filtered with measurement quality assessment, and t-statistics were calculated as discriminative weight values. After removing data points with no meaningful biological information and redundancy, we listed the most discriminatory genes (i.e., those genes which satisfy a weight greater than 4.0 and a group-wise averaged fold change (mean ratio difference between the two groups) greater than 2.0). Gene expression ratios were first log-transformed and then color-coded according to their standard deviation (σ) from the mean expression level across all experiments, with over-expression colored to red (dark gray) and under-expression to green (light green) (FIG. 1).

### Validation of Gene Expression Data by Real-Time RT-PCR

To validate the gene expression data obtained with DNA microarrays, we conducted relative real-time RT-PCR using total RNA from peripheral blood lymphocytes of additional patients (N=15) and female controls (N=15). All experiments were done in triplicates. In brief, total RNA was isolated from peripheral blood lymphocytes using the Trizol LS reagent (Invitrogen, Carlsbad, Calif.). To remove contaminating DNA, samples were treated with DNAse I (DNA-free, Ambion, Austin, Tex.). Reverse transcription was performed on the PTC-200 thermal cycler (MJ Research, Waltham, Mass.) using the iScript cDNA synthesis kit (Bio-Rad, Hercules, Calif.) following the manufacturer's protocol. After cDNA synthesis, PCR reactions were performed with specific oligo-primer pairs using the iQ SYBR Green Super Mix kit according to the manufacturer's recommendations (Bio-Rad, Hercules, Calif.). The PCR amplification profile was as follows: 94° C. for 4 min followed by 50 cycles of denaturation at 94° C./30 sec, gene-specific annealing temperature/30 sec, and extension at 72° C./40 sec. A melting curve was generated after each run to verify the specificity of the primers. Real-time analysis of PCR amplification was conducted using the iCycler iQ Optical System software, version 3.0a (Bio-Rad, Hercules, Calif.). Specific oligo-primer pairs were obtained from public databases and synthesized at the Molecular Resource Facility at New Jersey Medical School. Relative expression levels were calculated for each sample after normalization against the housekeeping gene GAPDH (Livak, K. J. 2001 Methods 25:402-408). Statistical analysis was performed using unpaired two-tailed t tests to compare relative mRNA expression levels in patients and controls (GraphPad InStat 3). Statistical significance was defined as a p value of <0.05.

### Example 2

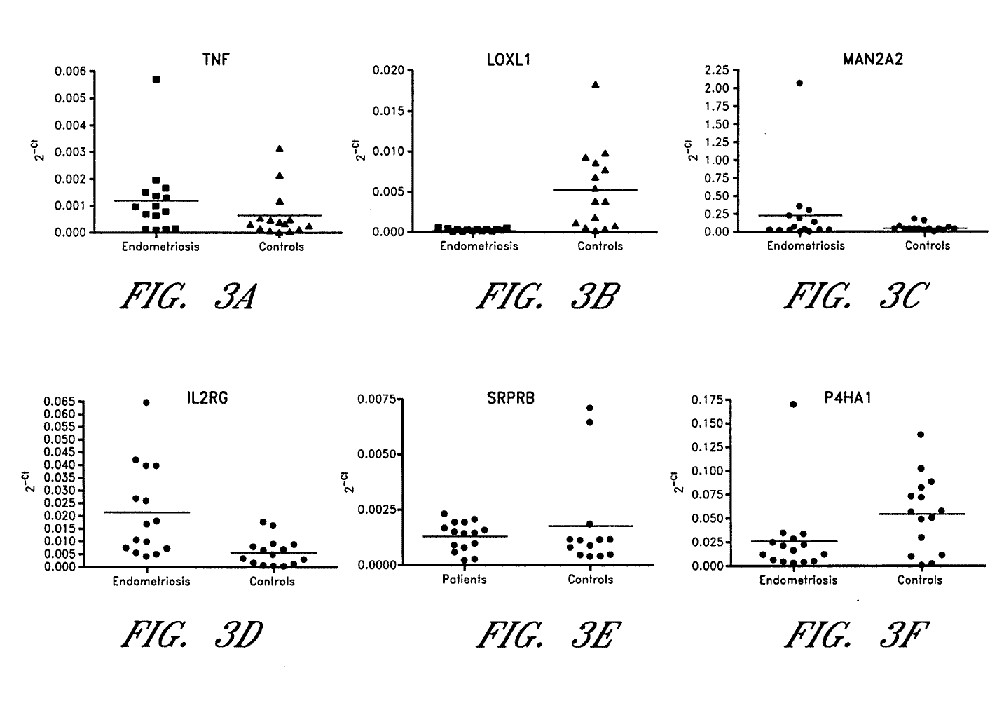

We completed analysis of data from 14 additional patients. We include scatter plots of the original data (Set 1) that allow a better representation of the individual variation of these markers (FIG. 3). Of note, scatter plots show that the low expression level of LOXL1 occurs in all the patients tested so far.

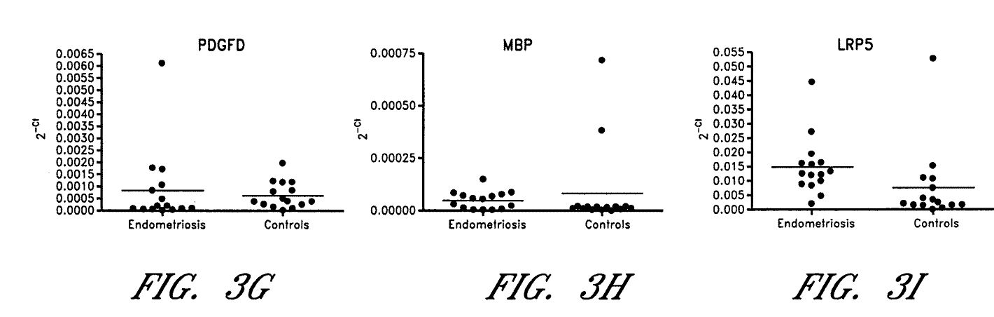

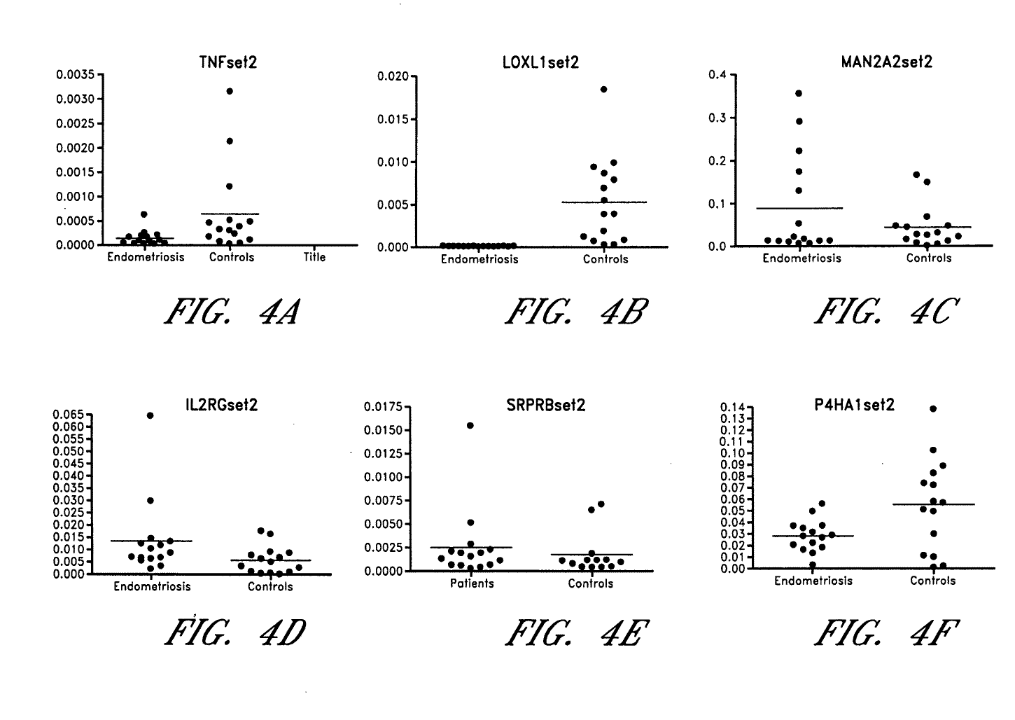

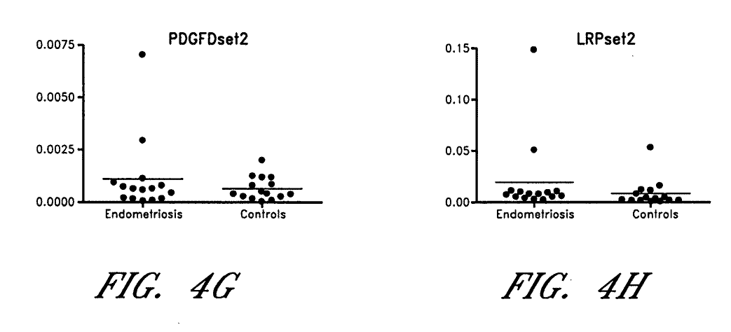

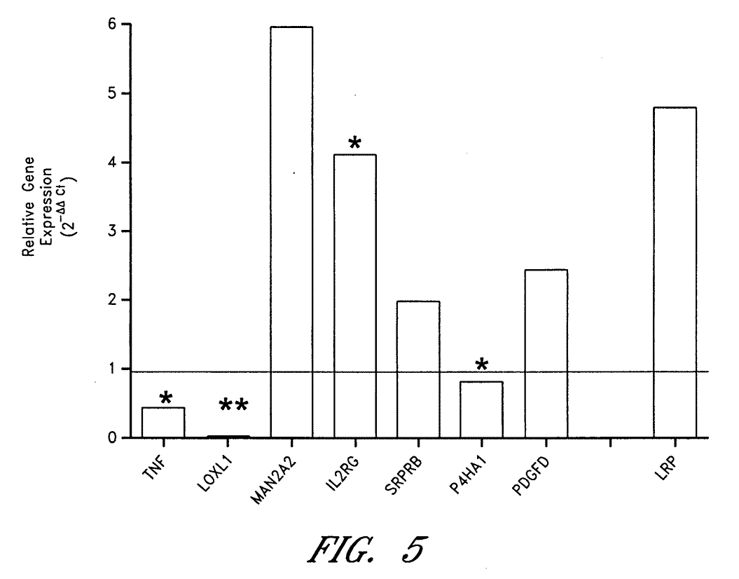

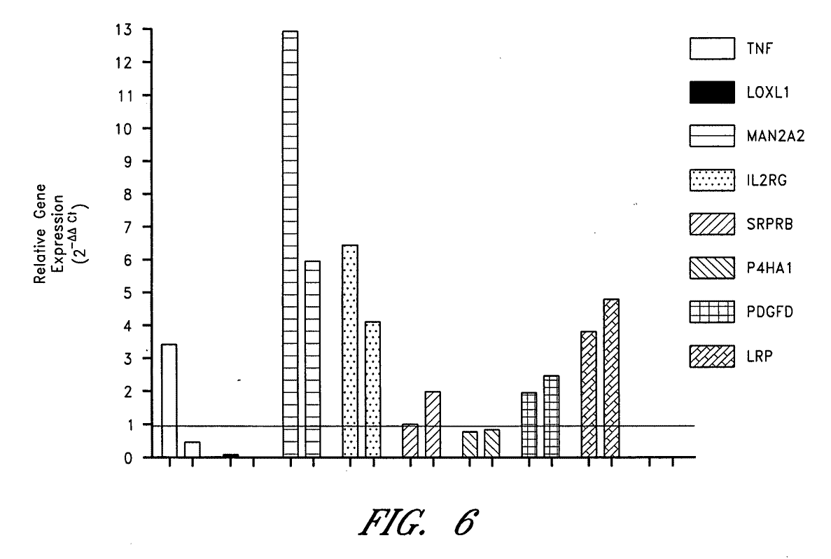

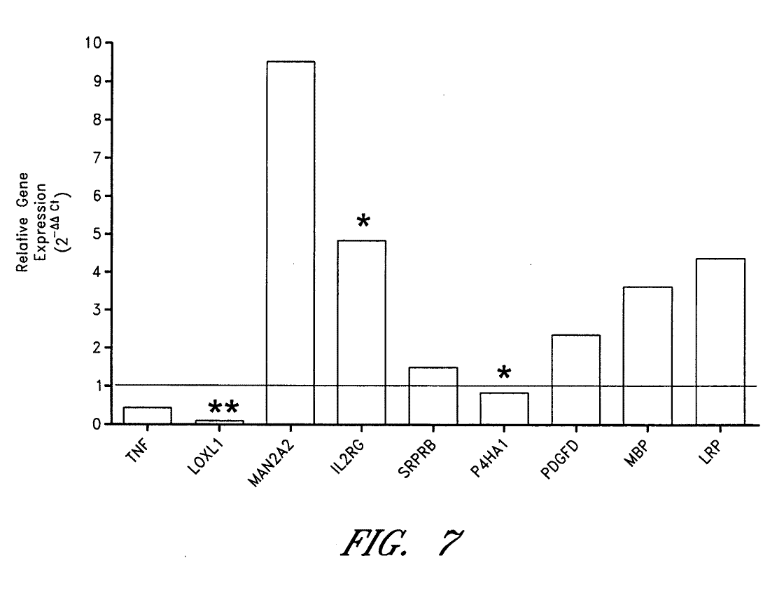

Also, we include additional data on another subset of 14 patients (Set 2) that confirms the original findings. The figures shown below present a summary of the results of the second set of patients studied. FIG. 4 consists of scatter plots showing individual variation of each gene for Set 2. FIG. 5 presents the averaged fold expression results from patients Set 1 (upper panel) and Set 2 (lower panel), while FIG. 6 shows a comparison of the results of the two groups. FIG. 7 plots the data from the 39 patients tested so far.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A method of identifying or predicting the predisposition to endometriosis in a female subject comprising:
(a) determining the level of gene expression of at least one differentially-expressed gene of peripheral blood leukocytes in a sample of peripheral blood leukocytes or peripheral blood in a subject to provide a first value,
(b) determining the level of gene expression of said at least one differentially-expressed gene of said leukocytes in a control or reference standard to provide a second value, and
(c) comparing whether there is a difference between said first value and second value.

2. A method according to claim 1 wherein said control or reference standard is determined from a subject or group of subjects without endometriosis.

3. A method according to claim 1 wherein the first value is greater than the second value is indicative of the presence or prediction of endometriosis.

4. A method according to claim 1 wherein the first value is lower than the second value is indicative of the presence or prediction of endometriosis.

5. A method according to claim 1 wherein the prediction of the presence of endometriosis has a probability of at least 50%.

6. A method according to claim 1 wherein the first value is at least 20% greater or lower than the second value.

7. A method according to claim 1 wherein determination of the level of gene expression comprises measuring the gene expression of a transcribed polynucleotide of the gene.

8. A method according to claim 7 wherein the transcribed polynucleotide is mRNA or cDNA.

9. A method according to claim 7 wherein the level of expression is detected by microarray analysis, Northern blot analysis, reverse transcription PCR or RT-PCR.

10. A method according to claim 1 wherein the level of gene expression of a member of the group consisting of LOXL1, IL2RG, LRP5, MPB, TNF, MAN2A2, P4HA1 and PDGF is determined.

11. A method of identifying or predicting the predisposition to endometriosis in a female subject comprising:
(a) determining the level of at least one differentially-expressed protein or peptide of peripheral blood leukocytes in a sample of peripheral blood leukocytes or peripheral blood in a subject to provide a first value,
(b) determining the level of said at least one differentially-expressed protein or peptide of said leukocytes in a control or reference standard to provide a second value, and
(c) comparing whether there is a difference between said first value and second value.

12. A method according to claim 11 wherein the first value is greater than the second value is indicative of the presence or prediction of endometriosis.

13. A method according to claim 11 wherein the first value is lower than the second value is indicative of the presence or prediction of endometriosis.

14. A method according to claim 11 wherein determination of the level of gene expression comprises measuring the protein expression product.

15. A method according to claim 11 wherein the amount of protein or peptide is detected using an antibody, antibody derivative or antibody fragment, which specifically binds to the protein.

16. A method of monitoring a subject identified as having endometriosis before and after treatment comprising:
(a) determining the level of gene expression of at least one differentially-expressed gene of peripheral blood leukocytes in a sample of peripheral blood leukocytes or peripheral blood in said subject prior to treatment providing a first value,
(b) determining the level of gene expression of at least one differentially-expressed gene of said leukocytes after treatment providing a second value, and
(c) comparing the difference in the level of gene expression of said subject before treatment and after treatment.

17. A method of screening candidate agents for use in treatment of endometriosis comprising:
(a) contacting a cell capable of expressing at least one differentially-expressed gene with a candidate agent ex vivo,
(b) determining the level of gene expression of said at least one differentially-expressed gene in said cell to provide a first value,
(c) determining the level of gene expression of the same at least one differentially-expressed gene in a cell in the absence of the candidate agent to provide a second value, and
(d) comparing the first value with the second value wherein a difference in the level of gene expression is indicative of an agent potentially capable of being used for the treatment of endometriosis.

18. A method of treating or preventing endometriosis comprising administering to a subject an effective amount of an agent that can induce a decrease or increase in the level of gene expression, synthesis, or activity of at least one differentially-expressed gene or gene expression product.

19. A method of manufacture of a medicament for the treatment or prevention of endometriosis comprising an effective amount of an agent that can induce a decrease or increase in the level of gene expression, synthesis, or activity of at least one differentially-expressed gene or gene expression product.

20. A kit for identifying or predicting the predisposition to endometriosis in a female subject comprising means for:
(a) determining the level of gene expression of at least one differentially-expressed gene of peripheral blood leukocytes in a sample of peripheral blood leukocytes or peripheral blood in a subject to provide a first value,
(b) determining the level of gene expression of said at least one differentially-expressed gene of said leukocytes in a control or reference standard to provide a second value, and
(c) comparing whether there is a difference between said first value and second value.
